(19) United States
(12) Patent Application Publication
(10) Pub. No.: US 2026/0158078 A1
(43) Pub. Date: Jun. 11, 2026

(54) Methods and Compositions for Managing and/or Alleviating Symptoms of Muscular Dystrophies and Other Genetic Muscular Disorders

(71) Applicant: MYOS CORP., Cedar Knolls, NJ (US)

(72) Inventors: Joseph MANNELLO, Cedar Knolls, NJ (US); Mary Catherine SABOL, Cedar Knolls, NJ (US)

(21) Appl. No.: 19/413,266

(22) Filed: Dec. 9, 2025

Related U.S. Application Data
(60) Provisional application No. 63/730,100, filed on Dec. 10, 2024.
(60) Provisional application No. 63/825,735, filed on Jun. 18, 2025.

(51) Int. Cl. A61K 35/57; A61P 21/00

(52) U.S. Cl. CPC A61K 35/57; A61P 21/00

(57) ABSTRACT

Methods for managing and/or alleviating one or more symptoms of a muscular dystrophy or other genetic condition associated with a muscular disorder via administering a composition of egg yolk derived product are provided.

This patent application claims the benefit of priority from U.S. Provisional Application Serial No. 63/825,735 filed Jun. 18, 2025 and U.S. Provisional Application Ser. No. 63/730,100 filed Dec. 10, 2024, the content of each of which is incorporated herein by reference in its entirety.

### BACKGROUND

Genetic muscular disorders are inherited conditions that that cause muscle weakness and dysfunction. Major categories of these genetic muscular disorders include muscular dystrophy, congenital myopathy and metabolic myopathy.

Some common genetic muscular disorders include Duchenne muscular dystrophy, Becker muscular dystrophy, myotonic muscular dystrophy, Facioscapulohumeral muscular dystrophy (FSHD), and congenital muscular dystrophy.

Duchenne muscular dystrophy (DMD) is one of the most severe inherited muscular diseases. Among hereditary neuromuscular diseases, DMD is the most common and it does not have a propensity towards any particular race or ethnic groups. The cause of DMD is a mutation on the Dystrophin gene located on chromosome Xp21 (Hoffman et al. Cell 1987 51(6 ):
919-928). The Dystrophin gene is one of the largest genes in the human genome, containing 79 exons of coding sequence with 2.5 Mb of DNA. The gene codes for the Dystrophin protein with a molecular weight (MW) of 427 kDa. DMD is inherited as an X-linked recessive trait but ˜30% of cases result from new mutations (Venugopal, V. and Pavlakis, S. “Duchenne Muscular Dystrophy.” 2018 StatPearls Publishing LLC). Because DMD is an X-linked disorder, it impacts primarily boys (Laing et al. The Clinical Biochemist Reviews 2011 32(3): 129). These mutations in the dystrophin gene lead to progressive muscle fiber degeneration and weakness by reducing the production of the dystrophin protein, thereby resulting in a loss of the membrane integrity of myofibers with repeated cycles of necrosis and regeneration (Venugopal, V and Pavlakis, S. “Duchenne Muscular Dystrophy.” 2018 StatPearls Publishing LLC). Over time, fibrous connective tissue and fat progressively replace muscle leading to the presentation of clinical features.

The weakness associated with DMD typically presents itself as difficulty with ambulation but eventually progresses and interferes with activities that are essential for daily life and causing patients to become wheelchair bound. It is common for patients that suffer from DMD to experience cardiac and orthopedic complications. Death commonly will occur during the second decade of life due to respiratory muscle weakness or cardiomyopathy.

For decades, the therapy for DMD has focused on treatment with glucocorticoids (Biggar et al. Neuromuscul Disord. 2006 16:249-55; Daftaryet al. Pediatrics. 2007 119: e320-4; Markham et al. Neuromuscul Disord. 2008 18:365-70) and physiotherapy in order to attenuate orthopedic complications.

In September 2016, the U.S. Food and Drug Administration (FDA) approved Eteplirsen, a phosphorodiamidate morpholino antisense oligonucleotide (PMO) that modulates splicing to treat DMD patients (Aartsma-Rus et al. Nucleic Acid Therapeutics 2017 27(1 ): 1-3).

Nutrition has been disclosed to play an important role in the management of DMD (Davis et al. Nutrition in Clinical Practice 2015 30(4): 511-521; Leighton, S. Nutrition and Dietetics 2003 60(1 ): 11-15; Willig et al. Developmental Medicine & Child Neurology 1993 35(12 ): 1074-1082).

Some encouraging results have been obtained in rodent studies involving green tea extract (Buetler et al. The American Journal of Clinical Nutrition 2002 75(4): 749-753 and Chinese herbal medicine (Chen et al. The American Journal of Chinese Medicine 2001 29(02 ): 281-292). The major polyphenol, epigallocatechin gallate isolated from green tea extract has been studied in the dystrophic mdx (5Cv) mouse model of DMD and was shown to lead to improvements in muscle function (Dorchies et al. American Journal of Physiology-Cell Physiology 2006 290(2): C616 -C625). Creatine monohydrate supplementation was has been shown to decrease cytoplasmic Ca²⁺ levels and increase intramuscular and cerebral phosphocreatine stores, providing potential musculoskeletal and neuroprotective effects in DMD Pearlman, J. P. and Fielding, R. A. Nutrition Reviews 2006 64(2 ): 80-88). Results from preclinical rodent studies suggest that the amino acids, taurine (De Luca et al. Journal of Pharmacology and Experimental Therapeutics 2003 304(1 ): 453-463) and glutamine (Granchelli et al. Neuromuscular Disorders 2000 10(4-5): 235-239) may offer some potential as it relates to the management of DMD. Recent research finding have also highlighted the potential of the flavanol, (−)-epicatechin in DMD due to promising improvements observed in preclinical rodent studies. (−)-Epicatechin is isolated from dark chocolate (cocoa) (Nogueira, Leonardo, et al. “(−)-Epicatechin enhances fatigue resistance and oxidative capacity in mouse muscle.” The Journal of physiology 589.18(2011 ): 4615-4631).

Becker muscular dystrophy is a milder form of muscular dystrophy that develops later in childhood than Duchenne.

Myotonic muscular dystrophy is the second most common muscular dystrophy, and can develop at any age. Life expectancy is not always affected, but severe cases can shorten life.

Facioscapulohumeral muscular dystrophy (FSHD) is the third most common muscular dystrophy, and can develop in childhood or adulthood. It progresses slowly and is not usually life-threatening.

Congenital muscular dystrophy is a genetic condition that affects muscle function due to a loss of glycoproteins in cells. This mutation increases the chance of muscles becoming injured and reduces their ability to repair damage.

Other genetic muscular disorders include, but are not limited to, Emery-Dreifuss muscular dystrophy, Limb-girdle muscular dystrophy, and Oculopharyngeal muscular dystrophy. Other inherited muscular disorders include congenital myopathies such as, but not limited to, nemaline myopathy, central core disease, and myotubular myopathy, myotonia congenita, and familial periodic paralysis. In addition, genetic conditions such as spinal muscular atrophy, Prader-Willi syndrome and other chromosome abnormalities, Pompe disease, Congenital myasthenia, Kennedy's disease, Amyotrophic lateral sclerosis, Zellweger syndrome spectrum and peripheral neuropathies, including, but not limited to, Guillain-Barré syndrome, are associated with muscular disorders.

Traditional muscle growth agents often fail in genetic muscular dystrophies and other genetic conditions leading to muscular disorders because they cannot overcome the core problem, e.g. the absence of a vital protein such as dystrophin in DMD or the presence of a toxic protein such as DUX4 in FSHD, which disrupt the normal muscle repair processes and promote muscle breakdown, inflammation, and replacement with fat and scar tissue.

There is a need for more effective treatments for managing and/or alleviating symptoms of muscular dystrophies and other genetic conditions leading to muscular disorders.

### SUMMARY

An aspect of the present invention relates to a method for managing and/or alleviating one or more symptoms of a muscular dystrophy or other genetic condition leading to a muscular disorder via administration of an effective amount of a composition comprising an egg yolk derived product.

In one nonlimiting embodiment, the composition comprising an egg yolk derived product is administered once, twice or three times a day.

In one nonlimiting embodiment, the composition comprises a fertilized egg yolk derived product.

In one nonlimiting embodiment, the fertilized egg yolk derived product is FORTETROPIN.

In one nonlimiting embodiment, administration of the egg yolk derived product alleviates weight loss associated with the muscular dystrophy or other genetic condition leading to a muscular disorder.

In one nonlimiting embodiment, administration of the egg yolk derived product alleviates muscle weakness associated with the muscular dystrophy or other genetic condition leading to a muscular disorder.

In one nonlimiting embodiment, the muscular dystrophy or other genetic muscular disorder is Duchenne muscular dystrophy.

### DETAILED DESCRIPTION

As demonstrated herein, administration of a composition comprising an egg yolk derived product unexpectedly promoted weight gain and increased strength as indicated by more hand use and overall mobility in an individual suffering from Duchenne muscular dystrophy after only three weeks of administration. From this demonstration, it is expected that administration of a composition comprising an egg yolk derived product will be similarly useful in managing and/or alleviating symptoms in other types of muscular dystrophy and other genetic muscular disorders.

Accordingly, the present invention provides methods and compositions for managing and/or alleviating one or more symptoms associated with muscular dystrophies and other genetic conditions associated with muscular disorders in a subject.

By “subject” it is meant to include any living organisms in which a muscular dystrophy and other genetic condition associated with a muscular disorder to be treated can occur.

Nonlimiting examples of subjects include mammals such as humans, apes, monkeys, cows, sheep, goats, dogs, cats, mice, rats, and transgenic species thereof.

By “managing and/or alleviating of one or more symptoms associated with a muscular dystrophy or other genetic muscular disorders” for purposes of this invention, it is meant to include, but is not limited to, inhibiting weight loss, promoting weight gain, increasing the number of muscle fibers, increasing the thickness of muscle fibers, increasing muscle mass, reducing oxidative stress-related damage to muscle fibers, improving mitochondrial function, reducing serum myostatin levels, reducing the concentration of inflammatory cytokines in serum, increasing the rate of muscle synthesis, increasing walking speed, increasing hand use, increasing hand grip strength in humans or force production in other mammals, increasing mobility, increasing ability of subject to perform daily hygiene activities without or with less assistance, improving respiratory function, reducing pain and discomfort and/or extending life span.

Further, the phrases “other genetic muscular disorder” and “other genetic condition associated with a muscular disorder” are used interchangeably herein and are meant to include any genetic conditions leading to muscle weakness and/or muscle loss in a subject one or more symptoms of which can be managed and/or alleviating in accordance with this disclosure.

Compositions administered in accordance with the present invention comprise egg yolk powder or liquid egg yolk or one or more proteins and/or lipids derived from egg yolk that are effective in managing and/or alleviating one or more symptoms associated with muscular dystrophies and other genetic conditions associated with muscular disorders in a subject.

In one nonlimiting embodiment, the composition comprises a fertilized egg yolk derived product.

In one nonlimiting embodiment, the composition administered is FORTETROPIN. FORTETROPIN is a fertilized egg yolk derived product used as a dietary and nutritional supplement (MYOS CORP., Cedar Knolls, NJ). Methods for production of FORTETROPIN are disclosed in U.S. Pat. Nos. 8,815,320, 10,165,785, and 11,051,524, teachings of each which are herein incorporated by reference in their entireties.

In one nonlimiting embodiment, production of a composition comprising an egg yolk derived product such as, but not limited to, FORTETROPIN is optimized to enhance potency as it relates to managing and/or alleviating one or more symptoms associated with muscular dystrophies and other genetic conditions associated with muscular disorders in a subject, by modifying one or more egg yolk-related parameters such as, but not limited to, incubation time post-lay, fertility status and breed of chicken.

In another nonlimiting embodiment, the composition comprises an avian follistatin such as described in U.S. Published Patent Application No. 2007/0275036, the disclosure of which is incorporated herein by reference in its entirety and/or other proteins and/or lipids found in avian eggs and which are beneficial in inhibiting weight loss, promoting weight gain and/or increasing muscle strength in subjects, and in particular humans, suffering from a muscular dystrophy or other genetic condition associated with a muscle disorder.

In another nonlimiting embodiment, the composition comprises a spray dried egg yolk powder or one or more proteins and/or lipids derived from egg yolk such as described in U.S. patent application Ser. No. 16/151,601, the disclosure of which is incorporated herein by reference in its entirety.

In one nonlimiting embodiment, the composition of egg yolk derived product comprises about 33% protein. Unlike high protein diets sometimes recommended to alleviate symptoms of muscular dystrophies such as Duchenne muscular dystrophy, which require careful planning to achieve 25-30 grams of protein in each meal, the egg yolk derived product has been shown to achieve results with one, two or possibly three times a day consumption of doses totaling about 8-9 grams of protein.

As the composition of egg yolk derived product is relatively low in calories at only 19.5 calories per 3-gram scoop, observed increases in weight gain are unlikely to be associated with increased caloric intake alone.

In one nonlimiting embodiment, the composition comprising the egg yolk derived product is administered orally on a daily basis in an amount ranging from about 1 to about 50 grams/day or about 10 to about 1000 mg/kg/day. In one nonlimiting embodiment, the dose is amount 15-30 grams/day. As will be understood by the skilled artisan upon reading this disclosure, amounts administered can be varied depending upon the severity of the muscle weakness, muscle loss and/or sarcopenia. Dosages may be modified for efficacy, for example, may be administered at a higher or lower dosage or administered more than once daily or less than once daily.

Dosing to the closest 12 scoop, or the like, can be performed without under dosing. In one nonlimiting embodiment, the composition is formed in a powder that may be mixed with other food to facilitate ingestion.

In one nonlimiting embodiment, the subject is administered FORTETROPIN orally as a powder on a daily basis in an amount ranging from about 15 to about 30 grams/day, or about 200 to about 500 mg/kg/day in humans. In canines and other nonhuman mammals, FORTETROPIN is administered orally as a powder on a daily basis in an amount ranging from about 200-1000 mg/kg/day.

However, as will be understood by the skilled artisan upon reading this disclosure, the compositions described herein can be formulated for administration as a powder or liquid to a mammal via any conventional means including, but not limited to, oral, or buccal.

Moreover, the compositions described herein, can be formulated into any suitable dosage form, including but not limited to, aqueous oral dispersions, liquids, gels, syrups, elixirs, slurries, suspensions and the like, for oral ingestion by an individual in need, solid oral dosage forms, controlled release formulations, fast melt formulations, effervescent formulations, lyophilized formulations, tablets, powders, pills, dragees, capsules, delayed release formulations, aqueous liquid dispersions, self-emulsifying dispersions, solid solutions, liposomal dispersions, solid dosage forms, powders, tablets, capsules, pills, delayed release formulations.

Formulations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including glucose, fructose, lactose, sucrose, mannitol, sorbitol, stevia extract, or sucralose; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Formulations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In some embodiments, the solid dosage forms disclosed herein may be in the form of a tablet, (including a suspension tablet, a fast-melt tablet, a bite-disintegration tablet, a rapid disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, pellets, granules. In other embodiments, the pharmaceutical formulation is in the form of a powder. In still other embodiments, the pharmaceutical formulation is in the form of a tablet. Additionally, formulations described herein may be administered as a single capsule or in multiple capsule dosage form. In some embodiments, the formulation is administered in two, or three, or four, capsules or tablets.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin-based film using technology and machinery known to those in the soft gel industry. The industrial units so formed are then dried to constant weight.

In some embodiments, the formulations may include other medicinal or pharmaceutical agents, carriers, diluents, dispersing agents, suspending agents, thickening agents, adjuvants, such as preserving, stabilizing, wetting or emulsifying agents, solution promoters, and/or buffers. In addition, the formulations can also contain other therapeutically valuable substances.

The formulations described herein can include an egg yolk derived product and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination(s) thereof. In still other aspects, using standard coating procedures, a film coating is provided around the formulation of the compound described herein. In one embodiment, some or all of the particles of the compound described herein are coated. In another embodiment, some or all of the particles of the compound described herein are microencapsulated. In still another embodiment, the particles of the compound described herein are not microencapsulated and are uncoated.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Formulations including an egg yolk derived product, as described herein, may be manufactured in a conventional manner, such as, by way of example only, by means of conventional mixing, dissolving, granulating, dragee-making, levigating, emulsifying, encapsulating, entrapping or compression processes.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations

Binders imparting cohesive qualities may also be used. Examples include, but are not limited to, alginic acid and salts thereof; cellulose derivatives such as carboxymethylcellulose, methylcellulose, hydroxypropylmethylcellulose, hydroxyethylcellulose, hydroxypropylcellulose, ethylcellulose, and microcrystalline cellulose; microcrystalline dextrose; amylose; magnesium aluminum silicate; polysaccharide acids; bentonites; gelatin; polyvinylpyrrolidone/vinyl acetate copolymer; crosspovidone; povidone; starch; pregelatinized starch; tragacanth, dextrin, a sugar, such as sucrose, glucose, dextrose, molasses, mannitol, sorbitol, xylitol, and lactose; a natural or synthetic gum such as acacia, tragacanth, ghatti gum, mucilage of isapol husks, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder.

Formulators skilled in art can determine the binder level for the formulations, but binder usage level of up to 70% in tablet formulations is common.

Compositions may further comprise carriers of relatively nontoxic chemical compounds or agents that facilitate the incorporation of a compound into cells or tissues. Nonlimiting examples include binders, suspending agents, disintegration agents, filling agents, surfactants, solubilizers, stabilizers, lubricants, wetting agents, diluents, and the like. Suitable carriers for use in solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Dispersing agents and/or viscosity modulating agents include materials that control the diffusion and homogeneity of a compound through liquid media or a granulation method or blend method. In some embodiments, these agents also facilitate the effectiveness of a coating or eroding matrix. Nonlimiting examples of diffusion/cilitators/dispersing agents include hydrophilic polymers, electrolytes, a Tween, PEG, polyvinylpyrrolidone, and carbohydrate-based dispersing agents such as hydroxypropyl celluloses (e.g., HPC, HPC-SL, and HPC-L), hydroxypropyl methylcelluloses (e. g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1, 1, 3, 3-tetramethylbutyl)-phenol polymer with ethylene oxide and formaldehyde (also known as tyloxapol), poloxamers, block copolymers of ethylene oxide and propylene oxide; and poloxamines, tetrafunctional block copolymers derived from sequential addition of propylene oxide and ethylene oxide to ethylenediamine, polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e. g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents that are particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

Compositions of the present invention may further comprise diluents used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose; dibasic calcium phosphate, dicalcium phosphate dihydrate; tricalcium phosphate, calcium phosphate; anhydrous lactose, spray-dried lactose; pregelatinized starch, compressible sugar; mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; sodium chloride; inositol, bentonite, and the like.

Compositions may further comprise an enteric coating, a substance that remains substantially intact in the stomach but dissolves and releases the egg yolk derived product in the small intestine or colon. Generally, the enteric coating comprises a polymeric material that prevents release in the low pH environment of the stomach but that ionizes at a higher pH, typically a PH of 6 to 7, and thus dissolves sufficiently in the small intestine or colon to release the active agent therein.

In addition, the compositions may comprise an erosion facilitator, a material that controls the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

Filling agents including compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like can also be included in the compositions. Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

In addition, flavoring agents and/or sweeteners can be used in the compositions and may include acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The compositions may further comprise lubricants and/or glidants that prevent, reduce or inhibit adhesion or friction of materials. Nonlimiting examples of lubricants include stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil, a surfactant, and the like.

Plasticizers, compounds used to soften the microencapsulation material or film coatings to make them less brittle may also be included in the compositions. Examples of suitable plasticizers include, but are not limited to, polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

The compositions may further comprise solubilizers such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In addition, the compositions my comprise stabilizers such as antioxidation agents, buffers, acids, preservatives and the like.

Suitable suspending agents for use in solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants including compounds such as sodium lauryl sulfate, sodium docusate, Tweens, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide and the like may also be included.

Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

Viscosity enhancing agents including, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof may also be included.

In addition, wetting agents including compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like may be included in these compositions.

In some embodiments, solid dosage forms, e.g., tablets, capsules, are prepared by mixing the egg yolk derived product described herein, with one or more pharmaceutical excipients to form a bulk blend composition. When referring to these bulk blend compositions as homogeneous, it is meant that the particles of egg yolk derived product are dispersed evenly throughout the composition so that the composition may be readily subdivided into equally effective unit dosage forms, such as tablets, pills, and capsules.

Conventional techniques include, e.g., one or a combination of methods: (1) dry mixing, (2) direct compression, (3) milling, (4) dry or non-aqueous granulation, (5) wet granulation, or (6) fusion. See, e. g., Lachman et al., “The Theory and Practice of Industrial Pharmacy” (1986).

It should be appreciated that there is considerable overlap between additives used in the solid dosage forms described herein. Thus, the above-listed additives should be taken as merely exemplary, and not limiting, of the types of additives that can be included.

A capsule may be prepared, for example, by placing the bulk blend of the formulation of the compound described above, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials which sufficiently isolate the compound from other non-compatible excipients. Materials compatible with the egg yolk derived product are those that delay the release of the egg yolk derived product in vivo.

In other embodiments, the formulations described herein, which include the egg yolk derived product, are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734.

In still other embodiments, the formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components. The enteric coated oral dosage form may also be a capsule (coated or uncoated) containing pellets, beads or granules of the solid carrier or the composition.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating for the methods and compositions described herein to achieve delivery to the lower gastrointestinal tract.

In some embodiments, formulations are provided that include particles of egg yolk derived product described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of egg yolk derived product, the liquid dosage forms may include additives, such as: (a) disintegrating agents; (b) dispersing agents; (c) wetting agents; (d) at least one preservative, (e) viscosity enhancing agents, (t) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists'Pharmacopeia (2005 edition, chapter 905), for at least 4 hours. The homogeneity should be determined by a sampling method consistent with regards to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

In one nonlimiting embodiment, the aqueous liquid dispersion can comprise a sweetening agent or flavoring agent in a concentration ranging from about 0.005% to about 0.5% the volume of the aqueous dispersion. In yet another embodiment, the aqueous liquid dispersion can comprise a sweetening agent or flavoring agent in a concentration ranging from about 0.01% to about 1.0% the volume of the aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1, 3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In some embodiments, the formulations described herein can be self-emulsifying drug delivery systems (SEDDS). Emulsions are dispersions of one immiscible phase in another, usually in the form of droplets. Generally, emulsions are created by vigorous mechanical dispersion.

SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art and include, but are not limited to, for example, U.S. Pat. Nos. 5,858,401, 6,667,048, and 6,960,563.

Buccal formulations that include egg yolk derived product may be administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period.

Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with the egg yolk derived product, and any other components that may be present in the buccal dosage unit. Generally, the polymeric carrier comprises hydrophilic (water-soluble and water-swellable) polymers that adhere to the wet surface of the buccal mucosa. Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

In certain embodiments, delivery systems for pharmaceutical compounds may be employed, such as, for example, liposomes and emulsions. In certain embodiments, compositions provided herein can also include a mucoadhesive polymer, selected from among, for example, carboxymethylcellulose, carbomer (acrylic acid polymer), poly (methylmethacrylate), polyacrylamide, polycarbophil, acrylic acid/butyl acrylate copolymer, sodium alginate and dextran.

The following nonlimiting examples are provided to further illustrate the present invention.

### EXAMPLE 1

### Assessing Efficacy in Duchenne Muscular Dystrophy

A 16 year old male suffering with Duchenne muscular dystrophy transitioned to a wheelchair 18 months ago and it was expected for his muscle function to steadily decline. However, he was administered FORTETROPIN daily at a dose of 26.4 grams/day. After only a 3 week period, he unexpectedly exhibited a gain in weight of about 5 to 6 pounds, which he has been unable to do previously for years, as well as an observable increase in use of his hands and the ability to move himself between his bed, wheelchair and toilet and to take his own shower. He has continued with his daily FORTETROPIN treatment and despite being in a wheelchair, he has continued to exhibit improvement and sustained strength in his legs, arms, and upper body over the past year, continuing to still get himself in and out of the shower and bed, dresses himself, and to manage his daily activities at home and at school with a level of independence that is unexpected at this

### EXAMPLE 2

### Assessing Muscle Size and Force in a Mouse Model of Mild FSHD

The objective of this study is to evaluate muscle function in dystrophic mice after 4 weeks (28 days) of administration of FORTETROPIN.

Male FLExDUX4. CRE mice were assigned to one of two groups based on body weight. Mice were treated with vehicle or FORTETROPIN (P.O. daily). Additionally, one group of littermate CRE WT mice served as reference and were treated with vehicle. After 4 weeks of treatment, mice underwent forced running endurance and muscle function testing, then the mice were euthanized, and tissues collected.

Forced running capacity was assessed using a treadmill run-to-exhaustion protocol. Mice were acclimated to the treadmill (LE 8710, Panlabs, Spain) for 3 days prior to the measurement. On the first day, mice were placed on the treadmill set at 0 m/min for 5 min at no inclination, then returned to their cage. The following two days, mice were placed on the treadmill set at 5 m/min for 5 min at no inclination, then returned to their cage. Following the acclimation period, mice were placed on the treadmill and the speed was set at 5 m/min for 5 min at no inclination, the speed was then increased by 5 m/min increments every 5 minutes until reaching 15 m/min, then increased by 2 m/min every 5 minutes until reaching 20 m/min and mice were run until exhaustion.

Following isoflurane sedation, muscle performance as measured in vivo with a 305C muscle lever system (Aurora Scientific Inc., Aurora, CAN). Mice were anesthetized via isoflurane inhalation (˜3-4.5%, or to effect) and placed on a thermostatically controlled table where anesthesia maintenance was via nose-cone (˜1-3% isoflurane, or to effect). The right knee was isolated using a pin pressed against the tibial head and the foot firmly fixed to a footplate on the motor shaft.

For the plantarflexor muscle group, contractions were elicited by percutaneous electrical stimulation of the sciatic nerve.

Optimal isometric twitch torque was determined by increasing the current with a minimum of 30 s between each contraction to avoid fatigue. A series of stimulations were then performed at increasing frequency of stimulation (0.2 ms pulse, 500 ms train duration): 1, 20, 40, 50, 60, 80, 100, 150 Hz. Maximal peak isometric force at each frequency, and force-frequency relationship was plotted.

Blood was collected via submandibular facial vein bleed. Briefly, mice were anesthetized and a 6 mm lancet was used to pierce the facial vein. Blood was collected into EDTA-coated capillary tube and centrifuged within 30 minutes to isolate plasma. Plasma was flash frozen then stored at −80° C. until shipping or disposition.

Following muscle function measurements and blood collection, animals were euthanized via anesthetic overdose followed by cervical dislocation and the gastrocnemius, tibialis anterior from both sides was collected. Muscles from the left leg were weighed and flash frozen.

The muscles from the right leg will be prepared by embedding in cryomatrix and frozen in 2-methylbutane cooled in dry ice. All tissues will be kept at −80° C. until shipping or disposition.

Treatment with FORTETROPIN resulted in an increase in muscle mass and an increase in running performance, without a change in muscle force production in this mouse model of mild

### EXAMPLE 3

### Assessing Efficacy in FSHD

A male in his upper sixties suffering from FSHD began taking FORTETROPIN daily at a dose of 39.6 grams/day. After approximately 5 weeks, he reported feeling more energy, better sleep, a change in heart rate value from low to normal and muscle content in his body going up. After two months on FORTETROPIN, a device worn in his shoe and often used by athletes to track injury recovery reported a general improvement over the last month inclusive of an increase in walking speed and Ground Contact Time Asymmetry and Impact Asymmetry trending towards zero (which is also an improvement).

What is claimed is:

1. A method for managing and/or alleviating one or more symptoms of a muscular dystrophy or other genetic condition associate with a muscular disorder, said method comprising administering to a subject in need an effective amount of a composition comprising an egg yolk derived product.

2. The method of claim 1, wherein the egg yolk derived product is administered up to three times a day.

3. The method of claim 1, wherein the composition comprises a fertilized egg yolk derived product.

4. The method of claim 4, wherein the fertilized egg yolk derived product is FORTETROPIN.

5. The method of claim 1, wherein administration of the egg yolk derived product alleviates weight loss associated with the muscular dystrophy or other genetic condition associated with a muscular disorder.

6. The method of claim 1, wherein administration of the egg yolk derived product alleviates muscle weakness associated with the muscular dystrophy or other genetic condition associated with a muscular disorder.

7. The method of claim 1, wherein the muscular dystrophy is Duchenne muscular dystrophy.

8. The method of claim 1, wherein the muscular dystrophy is Facioscapulohumeral muscular dystrophy (FSHD).

9. The method of claim 1, wherein the other genetic condition associated with a muscular disorder is spinal muscular atrophy.
